(19) United States
(12) Patent Application Publication
(10) Pub. No.: US 2021/0113437 A1
(43) Pub. Date: Apr. 22, 2021

(54) WIPE SUBSTRATE HAVING RESIDUAL DISINFECTANT PROPERTY

(71) Applicant: Microban Products Company, Huntersville, NC (US)

(72) Inventors: Gina Parise Sloan, Statesville, NC (US); Tian Lan, Huntersville, NC (US); Siqi Li, Charlotte, NC (US)

(21) Appl. No.: 17/070,476

(22) Filed: Oct. 14, 2020

Related U.S. Application Data
(60) Provisional application No. 62/916,965, filed on Oct. 18, 2019.

(51) Int. Cl. A61K 8/02; A61Q 17/00; A61K 8/34

(52) U.S. Cl. CPC A61K 8/0208; A61K 8/34; A61Q 17/005

(57) ABSTRACT

A wipe having a residual disinfectant property or efficacy is provided. A wipe having a residual disinfectant property provides an equal to or greater than 5 log reduction of bacteria even after multiple touches. The wipe has a substrate and a liquid formulation having a residual disinfectant property applied to the substrate. The liquid formulation is present in a ratio of weight of liquid formulation to weight of substrate in a range of 1:1 to 10:1. Preferably, the ratio of the weight of the liquid formulation to the weight of the wipe is 4:1 or <4:1.

### CROSS-REFERENCE TO RELATED APPLICATIONS

This application claims priority from U.S. Provisional Patent Application No. 62/916,965, filed on Oct. 18, 2019, in the United States Patent and Trademark Office. The disclosure of which is incorporated herein by reference in its entirety.

### FIELD OF THE INVENTION

The present invention relates to residual disinfectant products, more particularly to wipes having residual disinfectant properties.

### BACKGROUND OF THE INVENTION

A traditional disinfectant works immediately on contact; however, once touched, the surface is re-contaminated with skin organisms and potential pathogens. A residual disinfectant provides a kill immediately on contact and continues to provide disinfection even after repeat touches. Application via a spray or aerosol provides a format that is positioned to create a uniform film with no interfering compounds.

Placing the same residual formulation on a wipe construct or substrate introduces new hurdles. The wipe substrate itself could potentially absorb and sequester active ingredients possibly affecting efficacy. Additionally, the amount of liquid that is added to the wipe impacts efficacy and consumer use patterns. One must take into account absorptive capacity of the substrate, interfering wipe chemistries, excess liquid content, and wipe composition.

Thus, there is a need for a wipe product having a residual disinfectant property that overcomes the abovementioned disadvantages.

### SUMMARY OF THE INVENTION

In an embodiment of the invention, a substrate is provided in a form of a wipe having a residual disinfectant property. The wipe overcomes known problems associated with other disinfectant products.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### DESCRIPTION OF THE PREFERRED EMBODIMENTS

The following description of the embodiments of the present invention is merely exemplary in nature and is in no way intended to limit the invention, its application, or uses. The following description is provided herein solely by way of example for purposes of providing an enabling disclosure of the invention, but does not limit the scope or substance of the invention.

In an embodiment of the invention, a wipe having a residual disinfectant property or efficacy is provided. A residual disinfectant, as referred to in the context of the present invention, provides an equal to or greater than 5 log reduction of bacteria even after multiple touches.

A wipe having a residual disinfectant efficacy provides a unique benefit above and beyond a traditional disinfectant product. Traditional disinfectant products only provide disinfection efficacy immediately upon addition. A residual disinfectant product provides disinfection for an extended period of time following product application.

In an embodiment of the invention, the wipe comprises a natural material, a synthetic material, or a combination thereof. Examples of synthetic materials include, but are not limited to, polypropylene, polystyrene, polyester, nylon, rayon, acrylic, spandex, and a combination thereof. Examples of natural materials include, but are not limited to, wood, pulp, cellulose, cotton, linen, silk, wool, hemp, flax, and a combination thereof. Preferably, the wipe is comprised of a blend of synthetic materials.

The substrate may be woven or non-woven. A non-woven may be made by a variety of manufacturing methods including, but not limited to, melt-blown, conformed, spunbonded, air laid, hydroentangled, spun laced, bonded, carded, laminated or a combination thereof. The substrate may comprise a melt-blown polymeric material.

The wipe is treated with or contains a liquid formulation having a residual disinfectant property or efficacy. An example of a residual disinfectant formulation suitable for use in the present invention comprises a polymer binder, wherein the polymer binder is an oxazoline homopolymer, and a biocidal compound.

In an embodiment of the invention, the polymer binder is an oxazoline homopolymer. As another feature of the invention, the oxazoline homopolymer has the following structure:

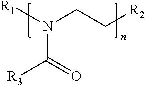

wherein

R₁ and R₂ are end groups determined by the polymerization techniques used to synthesize oxazoline homopolymer. R₁ and R₂ are independently selected and include, but are not limited to, hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether groups. Alternatively, R₂ could include a macrocyclic structure formed during synthesis as a consequence of intramolecular attack.

For example, R₁ is a methyl group and R₂ is oxazolinium tosylate if methyl tosylate is used as the initiator in the cationic initiated polymerization of oxazoline.

R₃ is an end group determined by the type of oxazoline used in the preparation of the polymer binder of this invention. R₃ includes, but is not limited to, hydrogen, alkyl, alkenyl, alkoxy, aryl, benzyl, hydroxyalkyl, or perfluoroalkyl. For example, R₃ is an ethyl group if ethyloxazoline is the monomer used to prepare the polymer binder for the present invention.

n is the degree of oxazoline polymerization in the homopolymer. n is in a range of 1 to 1,000,000. Preferably, n is in a range of 500 to 250,000; most preferably, n is in a range of 2500 to 100,000.

Examples of commercially available polyethyloxazolines include, but are not limited to, Aquazol 500 from Polymer Chemistry Innovations, Inc.

The amount of polymer binder that can be used in the liquid formulation can vary somewhat depending upon desired length of residual activity of the composition and the nature of all the other components in the composition. Preferably, the amount of polymer binder in the liquid formulation is in a range of 0.1% to 20% based on the weight of liquid formulation. In a liquid formulation for healthcare applications, the amount of polymer binder in the liquid formulation is more preferably in a range of 0.5% to 10%, and most preferably in a range of 0.8% to 5%. In liquid formulations for all-purpose and bathroom cleaners, the amount of polymer binder in the liquid formulation is more preferably in a range of 0.1% to 10%, and most preferably in a range of 0.1% to 5%.

The polymer binder preferably is water-soluble.

The biocidal compound may be a quaternary ammonium compound (QAC) with the following molecular structure:

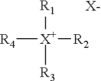

wherein

R₁, R₂, R₃, and R₄ are independently selected and include, but are not limited to, alkyl, alkoxy, or aryl, either with or without heteroatoms, or saturated or non-saturated. Some or all of the functional groups may be the same.

The corresponding anion X⁻ includes, but is not limited to, a halogen, sulfonate, sulfate, phosphonate, phosphate, carbonate/bicarbonate, hydroxy, or carboxylate.

QACs include, but are not limited to, n-alkyl dimethyl benzyl ammonium chloride, di-n-octyl dimethyl ammonium chloride, dodecyl dimethyl ammonium chloride, n-alkyl dimethyl benzyl ammonium saccharinate, and 3-(trimethoxysilyl) propyldimethyloctadecyl ammonium chloride.

Combinations of monomeric QACs are preferred to be used for the invention. A specific example of QAC combination is N-alkyl dimethyl benzyl ammonium chloride (40%); N-octyl decyl dimethyl ammonium chloride (30%); di-n-decyl dimethyl ammonium chloride (15%); and di-n-dioctyl dimethyl ammonium chloride (15%). The percentage is the weight percentage of individual QAC based on the total weight of blended QACs composition.

Still another polymeric QAC suitable for the invention is poly diallyldimethylammonium chloride or polyDADMAC.

Yet another class of QACs useful for the present invention are those chemical compounds with biguanide moiety in the molecule. Examples of this class of cationic antimicrobials include, but are not limited to, PHMB and chlorhexidine.

Examples of commercially available quaternary ammonium compounds include, but are not limited to, Bardac 205M and 208M from Lonza, and BTC885 from Stepan Company.

The biocidal compound may be a weak acid, which has been shown to be particularly effective in bathroom cleaners. In these type of products, citric, sulfamic (also known as amidosulfonic acid, amidosulfuric acid, aminosulfonic acid, and sulfamidic acid), glycolic, lactic, lauric and capric acids are useful as both an effective biocide and a cleaning agent for soap scum and hard wart deposits.

Other compounds which may be useful are silane quaternary salts such as 3(trihydroxysilyl)propyldimethyloctadecyl ammonium chloride. These may have the added benefit of reacting to the surface being treated for an enhancement of the residual properties.

Further biocidal compounds suitable for use in the present liquid formulation span a broad range of antimicrobials, biocides, sanitizers, and disinfectants. A water soluble or dispersible biocidal compound is preferred, although biocides soluble in alcohol may be alternatively employed.

A non-exhaustive list of biocidal compounds suitable for use in the present formulation include triclosan, zinc pyrithione, metal salts and oxides, phenols, botanicals, halogens, peroxides, heterocyclic antimicrobials, aldehydes, and alcohols.

The concentration of biocidal compound in the formulation can be in a range of 0.05% to 20% based on the weight of the liquid composition. For a liquid formulation for a healthcare application, preferably in a range of 0.1% to 20%, and more preferably in a range of 0.5% to 3%. For a liquid formulation for all-purpose and bathroom cleaners, preferably in a range of 0.05% to 10%. For a formulation for a protectant, preferably in a range of 0.05% to 2%.

The carrier or media for the liquid formulation of this invention can be any solvent that is volatile and allow easy evaporation at ambient condition. Examples of liquid carriers include, but are not limited to, water and low molecular weight alcohols such as C1 to C8 alkanols. Specific examples include, but are not limited to, ethanol, isopropyl alcohol, butanol, pentanol, and combinations thereof.

Another class of solvents for use in the invention includes alkylene glycol ether. Examples include, but are not limited to, ethylene glycol monopropyl ether, ethylene glycol monobutyl ether, ethylene glycol monohexyl ether, ethylene clycol monohexyl ether, diethylene glycol monomethyl ether, diethylene glycol monoethyl ether, diethylene glycol monobutyl ether, diethylene glycol monohexyl ether, triethylene glycol monomethyl ether, triethylene glycol monoethyl ether, triethylene glycol monobutyl ether, propylene glycol methyl ether, propylene glycol methyl ether acetate, propylene glycol n-butyl ether, dipropylene glycol n-butyl ether, dipropylene glycol methyl ether, dipropylene glycol methyl ether acetate, propylene glycol n-propyl ether, dipropylene glycol n-propyl ether, and tripropylene glycol methyl ether.

Another class of solvents for use in the invention is based on terpenes and their derivatives such as terpene alcohols, terpene esters, terpene ethers, or terpene aldehydes. Examples of solvents, include but are not limited to, pine oil, lemon oil, limonene, pinene, cymene, myrcene, fenchone, borneol, nopol, cineole, ionone and the like.

A surfactant or wetting agent may be employed. The surfactant assists the liquid formulation to spread and evenly coat the surface being treated.

In a preferred embodiment of the invention, the disinfection formulation imparting a residual biocidal property comprises a polyoxazoline having a structure of

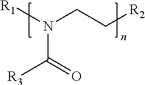

wherein: R₁ is methyl; R₂ is hydroxyl; R₃ is ethyl; and n=10 to 1,000,000, and

a quaternary ammonium compound (QAC) or a mixture of quaternary ammonium compounds, the QAC having a structure of

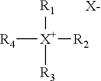

wherein

R₁ is methyl;

R₂ is alkyl C8-10; methyl-benzyl; or ethyl-benzyl;

R₃ is methyl;

R₄ is alkyl C8-18;

and

X⁻ is chloride or carbonate/bicarbonate.

In an embodiment of the invention, a method of making a wipe having a residual disinfectant efficacy is provided. The method comprises applying a liquid formulation having a residual disinfectant efficacy to a wipe. The liquid may be applied by padding, roller, or any other application mechanism known to one of ordinary skill in the art.

The liquid formulation is present in a ratio of weight of liquid formulation to weight of wipe substrate. Preferably, the weight of liquid formulation to weight of wipe substrate is in a range of 1:1 to 10:1. For example, the ratio of the weight of the liquid formulation to the weight of the wipe substrate is 5:1, preferably 4:1, and more preferably <4:1.

The active concentration of either the ethanol or the quaternary ammonium compounds in the liquid formulation can be measured before and after application to the wipe via any method capable of detecting quaternary ammonium compounds and/or ethanol. Examples include, but are not limited to, GC-MS for ethanol and an auto-titrator for quat. The active concentration of either the ethanol and/or the quaternary ammonium compound(s) typically does not change by more than 5% once applied to the wipe substrate. It is undesirable for the active in the concentration to bound and sequestered to the substrate.

The wipe substrate delivers 1 to 5 mg/cm² of liquid to a surface to create residual efficacy. This is measurable as weight deposition in mg of liquid formulation applied onto a surface.

The amount of liquid applied to the wipe can be altered if there is an increase in the viscosity of the liquid being applied. The viscosity of the liquid being applied is inversely proportional to the amount of liquid that is placed onto the wipe. An example of this is an increase in the polyoxazoline homopolymer to up to 10% of the weight of the liquid. The viscosity of the liquid is significantly increased with the increased concentration of the polyoxazoline and as such the load level on the wipe can be decreased by half to deliver the same efficacy.

Residual efficacy can be tested with a residual efficacy protocol available from EPA 01-1A.

### EXAMPLE

An example of a residual disinfectant formulation that was tested contained ethanol with a quaternary ammonium active ingredient, a surfactant, and a polyoxazoline polymer. It was identified that a dry film weighing 2 to 3 mg/in' delivered a residual disinfectant efficacy. The residual disinfectant formulation was applied using a spray product. All wipe substrates were first evaluated for deposition on a surface. It was determined that a product which contained a 50% or greater composition of a synthetic fiber, such as polyester (PET), provided optimal deposition of the formulation on the surface. The wipe with a majority of PET was able to be loaded at a 3:1 to 6:1 ratio of liquid formulation weight to wipe weight. Additionally, the synthetic nature of the wipe allowed for the robust release of active material, such as quaternary ammonium compounds, from the wipe. This corresponded to residual disinfectant activity on a surface.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A wipe having a residual disinfectant property, the wipe comprising:
a substrate, and
a liquid formulation having a residual disinfectant property applied to the substrate, wherein the wipe has an equal to or greater than 5 log reduction of bacteria after multiple touches.

2. The wipe according to claim 1, wherein the liquid formulation comprises ethanol.

3. The wipe according to claim 1, wherein the liquid formulation comprises a quaternary ammonium compound or a mixture of quaternary ammonium compounds.

4. The wipe according to claim 1, wherein the wipe delivers 1 to 5 mg/cm² of the liquid formulation to a surface to provide residual efficacy.

5. A wipe having a residual disinfectant property, the wipe comprising:
a substrate, and
a liquid formulation having a residual disinfectant property applied to the substrate, wherein the liquid formulation is present in a ratio of weight of liquid formulation to weight of substrate in a range of 1:1 to 10:1.

6. The wipe according to claim 5, wherein the liquid formulation comprises ethanol.

7. The wipe according to claim 5, wherein the liquid formulation comprises a quaternary ammonium compound or a mixture of quaternary ammonium compounds.

8. The wipe according to claim 5, wherein the ratio of the weight of the liquid formulation to the weight of the wipe is 5:1.

9. The wipe according to claim 5, wherein the ratio of the weight of the liquid formulation to the weight of the wipe is 4:1.

10. The wipe according to claim 5, wherein the ratio of the weight of the liquid formulation to the weight of the wipe is <4:1.

11. The wipe according to claim 5, wherein the wipe delivers 1 to 5 mg/cm² of the liquid formulation to a surface to provide residual efficacy.
